(19) United States
(12) Patent Application Publication
(10) Pub. No.: US 2008/0209591 A1
(43) Pub. Date: Aug. 28, 2008

(54) Mutant Allele of Cucurbita Pepo

(76) Inventor: Qi Zhang, Rocky Ford, CO (US)

Correspondence Address: JONDLE & ASSOCIATES P.C., 858 HAPPY CANYON ROAD SUITE 230, CASTLE ROCK, CO, 80108, US

(21) Appl. No.: 11/678,724

(22) Filed: Feb. 26, 2007

(51) Int. Cl. A01H 1/00; A01H 5/00; C12N 5/04

(52) U.S. Cl. 800/284; 435/410; 800/278; 800/279; 800/281; 800/310

(57) ABSTRACT

The present invention relates to a Cucurbita pepo plant, seed, variety and hybrid. More specifically, the invention relates to a C. pepo plant having a mutant allele designated HSPMR which results in a powdery mildew resistant plant. The invention also relates to crossing inbreds, varieties and hybrids containing the HSPMR allele to produce powdery mildew resistant C. pepo plants.

### BACKGROUND OF THE INVENTION

The present invention relates to a novel powdery mildew resistant allele of Cucurbita pepo L. designated “HSPMR”, which results in Cucurbita pepo plants having a strong powdery mildew resistance. The invention also relates to a C. pepo seed, a C. pepo variety and a C. pepo hybrid which contains the HSPMR allele. In addition, the present invention is directed to transferring the HSPMR allele to plants in the same species lacking the allele, and is useful for producing novel types and varieties of powdery mildew resistant Cucurbita pepo. All publications cited in this application are herein incorporated by reference.

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possesses the traits to meet the program goals. The goal is to combine in a single variety an improved combination of desirable traits from the parental germplasm. These important traits may include higher seed yield, resistance to diseases and insects, better stems and roots, tolerance to drought and heat, and better agronomic quality.

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to appropriate standards in environments representative of the commercial target area(s) for three or more years. The best lines are candidates for new commercial cultivars; those still deficient in a few traits may be used as parents to produce new populations for further selection.

These processes, which lead to the final step of marketing and distribution, usually take from eight to twelve years from the time the first cross is made. Therefore, development of new cultivars is a time-consuming process that requires precise forward planning, efficient use of resources, and a minimum of changes in direction.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

The goal of pumpkin and squash plant breeding is to develop new, unique and superior pumpkin and squash cultivars and hybrids. The breeder initially selects and crosses two or more parental lines, followed by repeated selfing and selection, producing many new genetic combinations. The breeder can theoretically generate billions of different genetic combinations via crossing, selfing and mutations. The breeder has no direct control at the cellular level. Therefore, two breeders will never develop the same line, or even very similar lines, having the same pumpkin and squash traits.

Each year, the plant breeder selects the germplasm to advance to the next generation. This germplasm is grown under unique and different geographical, climatic and soil conditions and further selections are then made during and at the end of the growing season. The cultivars that are developed are unpredictable because the breeder's selection occurs in unique environments with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce the same cultivar twice by using the same original parents and the same selection techniques. This unpredictability results in the expenditure of large amounts of research monies to develop superior new pumpkin and squash cultivars.

The development of new pumpkin and squash cultivars requires the development and selection of pumpkin and squash varieties, the crossing of these varieties and selection of superior hybrid crosses. The hybrid seed is produced by manual crosses between selected male-fertile parents or by using male sterility systems. These hybrids are selected for certain single gene traits such as fruit color, flower color, pubescence color or herbicide resistance which indicate that the seed is truly a hybrid. Additional data on parental lines, as well as the phenotype of the hybrid, influence the breeder's decision whether to continue with the specific hybrid cross.

Pedigree breeding and recurrent selection breeding methods are used to develop cultivars from breeding populations. Breeding programs combine desirable traits from two or more cultivars or various broad-based sources into breeding pools from which cultivars are developed by selfing and selection of desired phenotypes. The new cultivars are evaluated to determine which have commercial potential.

Pedigree breeding is used commonly for the improvement of self- and cross-pollinating crops. Two parents that possess favorable, complementary traits are crossed to produce an F₁. An F₂ population is produced by selfing one or several F₁s. Selection of the best individuals may begin in the F₂ population; then, beginning in the F₃, the best individuals in the best families are selected. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as the parents of commercial hybrids or new cultivars.

Mass and recurrent selections can be used to improve populations of either self- or cross-pollinating crops. A genetically variable population of heterozygous individuals is either identified, or created, by intercrossing several different parents or intracrossing in a heterogeneous population. The best plants are selected based on individual superiority, outstanding progeny, or excellent combining ability. The selected plants are intracrossed to produce a new population in which further cycles of selection are continued.

Backcross breeding has been used to transfer genes for a simply inherited, highly heritable trait into a desirable homozygous cultivar or inbred line which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In a multiple-seed procedure, pumpkin and squash breeders commonly harvest two or more seeds from the fruit of each plant in a population and bulk them to form a bulk sample. Part of the bulk is used to plant the next generation and part is put in reserve. The procedure has been referred to as modified single-seed descent or the “pod-bulk” (for bean crops) technique.

The multiple-seed procedure has been used to save labor at harvest. It is considerably faster to extract seeds with a machine than to remove one seed from each by hand for the single-seed procedure. The multiple-seed procedure also makes it possible to plant the same number of seeds of a population each generation of inbreeding. Enough seeds are harvested to make up for those plants that did not germinate or produce seed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Isozyme Electrophoresis and RFLPs have been widely used to determine genetic composition. Shoemaker and Olsen, (Molecular Linkage Map of Soybean (Glycine max L. Merr.) p 6.131-6.138 in S. J. O'Brien (ed) Genetic Maps: Locus Maps of Complex Genomes, Cold Spring Harbor Laboratory Press, Cold Spring Harbor, N.Y., (1993)) developed a molecular genetic linkage map that consisted of 25 linkage groups with about 365 RFLP, 11 RAPD, three classical markers and four isozyme loci. See also, Shoemaker, R. C., RFLP Map of Soybean, p 299-309, in Phillips, R. L. and Vasil, I. K., eds. DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. (Diwan, N. and Cregan, P. B., Theor. Appl. Genet. 95:22-225,1997.) SNPs may also be used to identify the unique genetic composition of the invention and progeny varieties retaining that unique genetic composition. Various molecular marker techniques may be used in combination to enhance overall resolution.

Molecular markers, which includes markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding is another method of introducing new traits into pumpkin and squash varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogues like 5-bromo-urcil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

Descriptions of other breeding methods that are commonly used for different traits and crops can be found in one of several reference books (e.g., Allard, 1960; Simmonds, 1979; Sneep et al., 1979; Fehr, 1987).

Proper testing should detect any major faults and establish the level of superiority or improvement over current cultivars. In addition to showing superior performance, there must be a demand for a new cultivar that is compatible with industry standards or which creates a new market. The introduction of a new cultivar will incur additional costs to the seed producer, the grower, processor and consumer for special advertising and marketing, altered seed and commercial production practices, and new product utilization. The testing preceding release of a new cultivar should take into consideration research and development costs as well as technical superiority of the final cultivar. For seed-propagated cultivars, it must be feasible to produce seed easily and economically.

The genus Cucurbita is composed of five species: C. pepo, C. maxima, C. moschta, C. mixta and C. ficifolia. The terms “pumpkin”, “squash” and “gourd” cannot be directly related to the species; forms of several are called pumpkins and the same is true of squashes and gourds. The term “pumpkin” is normally applied to the edible fruit of any species of Cucurbita utilized when ripe as a table vegetable, in pies, or as an ornamental. The term “squash” was evidently derived from a north-eastern American Indian word indicating a fruit, apparently Cucurbita pepo L., eaten raw as an immature fruit or consumed for the mature seed (T. W. Whitaker 1986. Breeding Vegetable Crops, pp. 210-223). It is now also applied to certain baking cultivars of C. pepo (e.g., Acorn), C. moschta (e.g., butternut), C. mixma (e.g., Orange Banana) and C. mixta (e.g., Cushaw) that are used in the mature state. The terms “pumpkin”, “squash” and “gourd” are confined to the species C. pepo. Pepo is an extremely large and diverse species in the genus Cucurbita which is separated into four categories: Halloween pumpkin, summer squash, winter squash and ornamental gourd. Each category is composed of many cultural types and has many varieties over the world. The pepo crops are widely cultivated and play a significant role in human nutrition and economic development globally.

The foregoing examples of the related art and limitations related therewith are intended to be illustrative and not exclusive. Other limitations of the related art will become apparent to those of skill in the art upon a reading of the specification.

### SUMMARY OF THE INVENTION

The following embodiments and aspects thereof are described in conjunction with systems, tools and methods which are meant to be exemplary, not limiting in scope. In various embodiments, one or more of the above-described problems have been reduced or eliminated, while other embodiments are directed to other improvements.

According to the invention, there is provided a novel mutant allele designated “HSPMR”. This invention thus relates to a C. pepo seed, a C. pepo plant, a C. pepo variety, a C. pepo hybrid, and to a method for producing a C. Pepo plant. More specifically, the invention relates to a mutant allele designated HSPMR which produces a C. pepo plant with powdery mildew resistance.

Another aspect of the invention relates to any C. pepo seed or plant having the mutant allele HSPMR.

In another aspect, the present invention provides regenerable cells for use in tissue culture. The tissue culture will preferably be capable of regenerating plants having the physiological and morphological characteristics of the foregoing C. pepo plant, and of regenerating plants having substantially the same genotype as the foregoing C. pepo plant. Preferably, the regenerable cells in such tissue cultures will be embryos, protoplasts, meristematic cells, callus, pollen, leaves, anthers, pistils, stems, petioles, roots, root tips, fruits, seeds, flowers, cotyledons, hypocotyls or the like. Still further, the present invention provides C. pepo plants regenerated from the tissue cultures of the invention.

Another aspect of the invention is to provide methods for producing other C. pepo plants derived from a C. pepo plant having the HSPMR allele. C. pepo lines derived by the use of those methods are also part of the invention.

The invention also relates to methods for producing a C. pepo plant containing in its genetic material one or more transgenes and to the transgenic C. pepo plant produced by that method.

The invention further provides methods for developing C. pepo plants in a C. pepo plant breeding program using plant breeding techniques including recurrent selection, backcrossing, pedigree breeding, restriction fragment length polymorphism enhanced selection, genetic marker enhanced selection and transformation. C. pepo seeds, plants and parts thereof produced by such breeding methods are also part of the invention.

In addition to the exemplary aspects and embodiments described above, further aspects and embodiments will become apparent by study of the following descriptions.

### DEFINITIONS

In the description and tables that follow, a number of terms are used. In order to provide a clear and consistent understanding of the specification and claims, including the scope to be given such terms, the following definitions are provided:

Allele. An “allele” is any of one or more alternative form of a gene, all of which alleles relates to one trait or characteristic. In a diploid cell or organism, the two alleles of a given gene occupy corresponding loci on a pair of homologous chromosomes.

Backcross. “Backcross” is a breeding method in which a breeder repeatedly crosses hybrid progeny back to one of the parents, for example, a first generation hybrid F₁ with one of the parents of the F₁ hybrid.

Complete growing season. “Complete growing season” means from the C. pepo planting date until 95% of C. pepo leaves have died naturally and not due to disease.

Consistent resistance. “Consistent resistance” refers to plants expressing resistance to powdery mildew for the complete growing season when powdery mildew is present in the environment.

Essentially all the physiological and morphological characteristics. A plant having “essentially all the physiological and morphological characteristics” means a plant having the physiological and morphological characteristics, except for the characteristics derived from the converted gene.

HSPMR. “HSPMR” refers to the mutant allele of the present invention that confers powdery mildew resistance to C. pepo plants.

Pedigree breeding/selection. “Pedigree breeding” is a breeding method used during the inbreeding of populations of self- and cross-pollinated species for the development of desirable homogeneous lines. Pedigree selection generally begins with an F₂ population and continues until homogeneous lines are developed.

Plant. “Plant” includes plant cells, plant protoplasts, plant cells of tissue culture from which C. pepo plants can be regenerated, plant calli, plant clumps and plant cells that are intact in plants, or parts of plants such as pollen, flowers, seeds, leaves, stems, rind, flesh and the like.

PMR. “PMR” refers to a C. pepo plant phenotypically displaying a level of powdery mildew resistance. The level of powdery mildew on plants is divided into six categories consisting of 0, 1, 2, 3, 4, 5, in which 0 indicates that the plants are completely resistant to the disease and there are no symptoms of the disease on the plants at all, and a rating of 5 indicates that the plants are 100% infected and susceptible.

PMS. “PMS” refers to a C. pepo plant phenotypically displaying powdery mildew susceptibility.

Quantitative Trait Loci (QTL). “Quantitative trait loci (QTL)” refer to genetic loci that control to some degree numerically representable traits that are usually continuously distributed.

Regeneration. “Regeneration” refers to the development of a plant from tissue culture.

Single gene converted (conversion). “Single gene converted” (or conversion) plant refers to plants which are developed by a plant breeding technique called backcrossing wherein essentially all of the desired morphological and physiological characteristics of an inbred are recovered in addition to the single gene transferred into the inbred via the backcrossing technique or via genetic engineering.

### DETAILED DESCRIPTION OF THE INVENTION

The present invention relates to a novel mutant allele designated “HSPMR” in the genus Cucurbita that is phenotypically expressed in powdery mildew resistance. As used herein powdery mildew resistance refers to plants that display little to no disease symptoms throughout the growing season.

According to the invention, there is provided a novel mutant allele designated “HSPMR”. This invention thus relates to a C. pepo seed, a C. pepo plant, a C. pepo variety, a C. pepo hybrid, and to a method for producing a C. pepo plant. More specifically, the invention relates to a mutant allele designated HSPMR which produces a C. pepo plant with powdery mildew resistance.

Another aspect of the invention relates to any C. pepo seed or plant having the mutant allele HSPMR.

A common disease of C. pepo crops is powdery mildew which is largely caused by two different fungi: Sphaerotheca fuliginea Poll. and Erysiphe cichoracearum DC. Powdery mildew epidemics are ubiquitous and often result in considerable losses to fields of pumpkin, squash and gourd. Powdery mildew infections on the crops appear as a white powdery growth that develops on the leaf blades, peduncles, and stems of susceptible plants. Usually infection begins on older leaves and then spreads to other parts of the plant. It frequently compromises yield by reducing the leaf area available for photosynthesis, accelerating senescence, and, ultimately, killing the infected plants. Therefore, the disease is one of the main limiting factors of pumpkin, squash and gourd production around the world.

To solve the powdery mildew disease problem, breeding for powdery mildew resistance has been conducted world-wide for a number of years. The most common commercially available pumpkin and squash varieties have powdery mildew resistance which is conferred by a single co-dominant gene (See M. T. Mcgrath. 2004. Managing powdery mildew in winter squash with genetic control and chemical control. Plant Pathology, Cornell Univ. Pub. No. P-2004-0016-Nea; and R. Cohen et. al. 2004. Single-gene resistance to powdery mildew in zucchini squash (Cucurbita pepo L.) Euphytica, Vol.130 (3):433-441). The heterozygous plants carrying this co-dominant gene produce a segregating ratio of 1 powdery mildew resistant plant to 2 powdery mildew tolerant plants to 1 powdery mildew susceptible plant. C. pepo varieties currently available exhibit a range in the level of resistance depending on whether the variety is heterozygous or homozygous for the co-dominant gene. None of the currently available varieties keep a consistent powdery mildew resistance level during the complete growing season. Varieties homozygous for currently available co-dominant gene usually develop somewhat less powdery mildew than those heterozygous for currently available co-dominant gene. In catalogues these varieties are often described as resistant and tolerant, respectively.

All currently available pumpkin and squash varieties designated as being powdery mildew resistant or tolerant exhibit some suppression of powdery mildew development for a few weeks, but become affected by different levels of disease severity and may die up to a month or more before the end of the complete growing season. Effective powdery mildew chemical control is therefore still needed in order to get fruit and seed production. Although chemicals such as Tosin M, Nova, Flint, Amistar, Quadris, Quintec, DMI, Baylaeton, Procure etc. have been used to effectively manage powdery mildew for decades, unfortunately, all such materials are at risk for the development of resistance by the various powdery mildew strains. In view of the inevitable occurrence of powdery mildew and the significant expense for disease control, breeding for better powdery mildew resistance in pumpkin and squash is very important.

The genetic factor of the present invention which is capable of transmitting powdery mildew resistance has been determined to be a single dominant allele which has been designated “HSPMR”. It is a feature of the present invention that this single mutant allele HSPMR may be used in and transferred among the various pumpkin, squash and gourd varieties in the C. pepo species.

In another aspect, the present invention provides regenerable cells for use in tissue culture. The tissue culture will preferably be capable of regenerating plants having the physiological and morphological characteristics of the foregoing C. pepo plant, and of regenerating plants having substantially the same genotype as the foregoing C. pepo plant. Preferably, the regenerable cells in such tissue cultures will be embryos, protoplasts, meristematic cells, callus, pollen, leaves, anthers, pistils, stems, petioles, roots, root tips, fruits, seeds, flowers, cotyledons, hypocotyls or the like. Still further, the present invention provides C. pepo plants regenerated from the tissue cultures of the invention.

Another aspect of the invention is to provide methods for producing other C. pepo plants derived from a C. pepo plant having the HSPMR allele. C. pepo lines derived by the use of those methods are also part of the invention.

The invention also relates to methods for producing a C. pepo plant containing in its genetic material one or more transgenes and to the transgenic C. pepo plant produced by that method.

In another aspect, the present invention provides for single gene converted plants of HSPMR. The single transferred gene may preferable be a dominant or recessive allele. Preferably, the single transferred gene will confer such trait as male sterility, herbicide resistance, insect resistance, resistance for bacterial, fungal, or viral disease, male fertility, improved harvest characteristics, enhanced nutritional quality, and improved processing characteristics. The single gene may be a naturally occurring C. pepo gene or a transgene introduced through genetic engineering techniques.

The invention further provides methods for developing C. pepo plants in a C. pepo plant breeding program using plant breeding techniques including recurrent selection, backcrossing, pedigree breeding, restriction fragment length polymorphism enhanced selection, genetic marker enhanced selection and transformation. C. pepo seeds, plants and parts thereof produced by such breeding methods are also part of the invention.

The present invention is directed to developing unique plants of the Cucurbita species. The pumpkin fruit and plants of the present invention unexpectedly express a substantial increase in powdery mildew resistance. A transferable gene or allele that conveys this characteristic has been isolated and incorporated into other genetic backgrounds. The allele of the instant invention has also been expressed in different genetic backgrounds of pumpkin. To date, no commercialized C. pepo variety has the consistent resistance to powdery mildew conferred by the mutant allele HSPMR of the present invention. The crosses with the mutant allele HSPMR of the present invention unexpectedly expressed a unique resistance pattern during the last two years of trials, in which the plants showed a consistent strong resistance level throughout the whole growing season and without the aid of any chemical application. It would be commercially very desirable to have new varieties of C. pepo that have a better and more consistent resistance to powdery mildew for growers, the commercial market, and especially for organic growers.

As used herein, the term plant includes plant cells, plant protoplasts, plant cell tissue cultures from which C. pepo plants can be regenerated, plant calli, plant clumps and plant cells that are intact in plants, or parts of plants, such as embryos, pollen, ovules, flowers, stems, leaves, roots, root tips, anthers, pistils, and the like.

### EXAMPLES

The following examples are provided to further illustrate the present invention and are not intended to limit the invention beyond the limitations set forth in the appended claims.

### Example 1

### Development of HSPMR, the Mutant Allele of the Present Invention.

The mutant allele of the present invention, HSPMR, unexpectedly arose from an open pollinated pumpkin plant from a research field in 2000. The series of crosses and selections is shown in Table 1. [(Howden×Coach)×Sprit] in Table 1 was a pumpkin three-way cross made between 1999 and 2000. None of these three parents have the resistance of the allele of the present invention. The H7B-1Lop population, considered an F₂, was derived from a plant which exhibited strong powdery mildew resistance that was open-pollinated in the three-cross generation in the summer of 2000. During a pedigree selection process, five plants which exhibited a high level of powdery mildew resistance were selected and selfed from the F₂ population in 2001 and used to form the F₃ families H7B-1LOP-1, H7B-1 LOP-2, H7B-1LOP-3, H7B-1LOP-4, and H7B-1LOP-1LOP-3-5, respectively. In the summer of 2002, it was found that families H7B-1LOP-2, H7B-1LOP-3and H7B-1LOP-5 all produced ratios of 3 powdery mildew resistant plants to 1 powdery mildew susceptible plant, while all the plants in the H7B-1LOP-1 family were powdery mildew resistant. Four powdery mildew plants in the H7B-1Lop-1 family and one in the H7B-1Lop-4 family were further selfed and used to form the H7B-1Lop-1-1, H7B-1Lop-1-2, H7B-1Lop-1-3, H7B-1Lop-1-4 and H7B-1Lop-4-1 families. The F₄ seeds were planted and the seedlings were screened with powdery mildew inocula (Sphaerotheca fuliginea Poll. and Erysiphe cichoracearum DC). The powdery mildew screening showed that all five families were nonsegregating for the powdery mildew resistance trait in the fall of 2002. The F₅ lines, H7B-1Lop-1-1-1, H7B-1Lop-1-1-2, H7B-1Lop-1-1-3 and H7B-1Lop-4-1-1 were produced from the F₄ families H7B-1Lop-1-1 and H7B-1Lop-4-1 in the same season. At the same time two crosses were also made by crossing F₄ (H7B-1Lop-1-1) plants to a common powdery mildew susceptible pumpkin male designated 710M. Additionally, two backcrosses (BC) were made to produce BC populations for further testing during the winter of 2002. The backcrosses were (H7B-1Lop-1-1×710M)BC11 and (H7B-1Lop-1-1×710M)BC12.

Table 1 shows a summary of the series of crosses described in the previous paragraph. Column 1 shows the pedigree, column 2 shows the year for each cross and column 3 shows remarks concerning each cross.

TABLE 1

| Pedigree | Year | Remarks |
|---|---|---|
| Recombining Generation | | |
| [(Howden × Coach) × Sprit]-1L | 1999–2000 | Designated H7B-1L |
| O.P. or F2 Population | | |
| H7B-1Lop | 2001 | O.P. plant with strong |
| | | PMR |
| F₃ Family | | |
| H7B-1Lop-1 | 2002 | No PMR Segregation |
| H7B-1Lop-2 | 2002 | PMR segregation, |
| | | 3PMR:1PMS |
| H7B-1Lop-3 | 2002 | PMR segregation, |
| | | 3PMR:1PMS |
| H7B-1Lop-4 | 2002 | No PMR segregation |
| H7B-1Lop-5 | 2002 | PMR segregation, |
| | | 3PMR:1PMS |
| F₄ Family | | |
| H7B-1Lop-1-1 | 2002 | No PMR segregation |
| H7B-1Lop-1-2 | 2002 | No PMR segregation |
| H7B-1Lop-1-3 | 2002 | No PMR segregation |
| H7B-1Lop-1-4 | 2002 | No PMR segregation |
| H7B-1Lop-4-1 | 2002 | No PMR segregation |
| F₅ Family | | |
| H7B-1Lop-1-1-1 | 2003 | No PMR segregation |
| H7B-1Lop-1-1-2 | 2003 | No PMR segregation |
| H7B-1Lop-1-1-3 | 2003 | No PMR segregation |
| H7B-1Lop-4-1-1 | 2003 | No PMR segregation |
| Test Crosses & Backcrosses | | |
| H7B-1Lop-1-1 × 710M | 2002 | |
| (H7B-1Lop-1-1 × 710M)BC11 | 2002 | PMR segregation, |
| | | 1PMR:1PMS |
| (H7B-1Lop-1-1 × 710M)BC12 | 2002 | PMR segregation, |
| | | 1PMR:1PMS |
| F₁ Hybrids | | |
| H7B-1Lop-1-1-1 × BSP1 | 2002 | Designated HSR4706, |
| | | PMR |
| H7B-1Lop-1-1-1 × GDF41 | 2002 | Designated HSR4707, |
| | | PMR |
| H7B-1Lop-1-1-1 × HDD31 | 2002 | Designated HSR4709, |
| | | PMR |

### Example 2

### Determining the Genetic Mechanism of Powdery Mildew Resistance of the Present Invention

To determine the genetic mechanism of the new powdery mildew resistance characteristic of the present invention, five F₃ families were produced and planted in the summer of 2002. These five F₃ families were produced by selecting and selfing five plants from the F₂ population in 2001. The F₃ families produced from the selected five plants were designated H7B-1LOP-1, H7B-1LOP-2, H7B-1LOP-3, H7B-1LOP-4, and H7B-1LOP-5, respectively. It was found that families H7B-1LOP-2, H7B-1LOP-3 and H7B-1LOP-5 all produced ratios of 3 powdery mildew resistant plants to 1 powdery mildew susceptible plant however plants in the H7B-1LOP-1 family were all powdery mildew resistant. Upon comparison of comprehensive characteristics among the families, it was found that families H7B-1Lop-2, H7B-1Lop-3 and H7B-1Lop-5 were not ideal in either fruit traits or plant habits and thus, these families were not used in further breeding efforts.

The two remaining families, H7B-1Lop-1 and H7B-1Lop-4, had desirable fruit characteristics, therefore four plants of the H7B-1Lop-1 family and one plant of the H7B-1Lop-4 family were selected and selfed to form H7B-1Lop-1-1, H7B-1Lop-1-2, H7B-1Lop-1-3, H7B-1Lop-1-4 and H7B-1Lop-4-1, respectively. The resulting F₄ seeds were planted and the seedlings were screened with powdery mildew inocula (Sphaerotheca fuliginea Poll. and Erysiphe cichoracearum DC). The powdery mildew screening showed that all five F₄ families were non-segregating for the powdery mildew resistance trait in the fall of 2002. Of the F₄ families, two were kept for advanced selections of other traits except for powdery mildew. The F₅ families, H7B-1Lop-1-1-1, H7B-1Lop-1-1-2, H7B-1Lop-1-1-3 and H7B-1Lop-4-1-1 were produced from the 2002 season. At the same time two crosses were also made by crossing F₄ (H7B-1Lop-1-1) plants to a common powdery mildew susceptible pumpkin male designated 710M. Additionally, two backcrosses (BC) were made to produce BC populations for further testing during the winter of 2002. The backcrosses were (H7B-1Lop-1-1×710M)BC11 and (H7B-1Lop-1-1×710M)BC12. The two BC populations were tested and both segregated 1 powdery mildew resistant plant to 1 powdery mildew susceptible plant during the summer 2003.

The data, as shown in Table 2, indicated that, except for the H7B-1LOP-1 family, all four other families had a heterozygous genotype and that the powdery mildew resistance phenotype of the present invention is under the control of a single dominant allele designated HSPMR.

Table 2 shows the genetic mechanism of the new powdery mildew characteristic. Column 1 shows the pumpkin family, column 2 shows the number of plants in that family displaying the powdery mildew resistant phenotype, column 3 shows the number of plants in that family displaying the powdery mildew susceptible phenotype and columns 4 and 5 show the Chi-square analysis of the data.

TABLE 2
PMR segregation in six selfed and BC families

| | | | Chi-square^{z} | |
|---|---|---|---|---|
| Family | PMR | PMS | χ²(3:1) | χ²(1:1) |
| F₃ Families | | | | |
| H7B-1Lop-1 | 55 | 0 | — | — |
| H7B-1Lop-2 | 37 | 13 | 0.0267 | — |
| H7B-1Lop-3 | 47 | 16 | 0.0053 | — |
| H7B-1Lop-4 | 28 | 9 | 0.0090 | — |
| H7B-1Lop-5 | 31 | 11 | 0.032 | — |
| BC Populations | | | | |
| (H7B-1Lop-1-1 × 710M)BC11 | 19 | 17 | — | 0.1111 |
| ((H7B-1Lop-1-1 × 710M)BC12 | 24 | 27 | — | 0.1765 |

^{Z}All χ² are non significant at 0.01 probability and the PMR and PMS segregation meet 3:1.
— means not tested.

### Example 3

### Assessing the Level of Powdery Mildew Resistance Conferred by the Mutant Allele HSPMR of the Present Invention in Hybrid Form

To assess the powdery mildew resistance level conferred by the mutant allele HSPMR of the present invention in hybrid form, three selected commercially available powdery mildew susceptible pumpkin lines BSP1, GDF41, and HDD31 were crossed with the powdery mildew resistant line H7B-1Lop-1-1-1 which contains the mutant allele HSPMR of the present invention, respectively, and formed three hybrids, HSR4706, HSR4707 and HSR4709 in 2004. These hybrids each contain the mutant allele HSPMR of the present invention and were tested for powdery mildew resistance successively in experimental trials during the summers of 2005 and 2006. The level of powdery mildew on plants was divided into six scores or fractions thereof consisting of 0, 1, 2, 3, 4, 5, in which 0 indicates that the plants were completely resistant to the disease and there were no symptoms of the disease on the plants at all, and a rating of 5 indicates that the plants were 100% infected and susceptible. Increasing one level among the six scores means that the resistance to powdery mildew disease decreased by 20% or, conversely, the susceptibility to the disease increased by 20%. For example, a plant with a score of 1 means that plant showed 20% more disease symptoms than a plant with a score of 0. In another example, a plant with a score of 3 means that plant shows 60% more disease symptoms than a plant with a score of 0. Each score is based on the following: the ratio of infected leaves and stems on a plant, the proportion of infected area on a leaf and a stem, and the colony counts out of the total in the infected area. To better understand the difference among the powdery mildew resistance levels, a significance test reciprocal transformation was applied during the mean separation process.

The first powdery mildew resistance pumpkin hybrid trial was conducted in the summer of 2005. The experiment was designed to determine whether the new powdery mildew resistance characteristic conferred by the mutant allele HSPMR of the present invention in hybrids is superior to that of what is available in commercial pumpkin varieties. The new hybrids containing the mutant allele HSPMR were HSR4706, HSR4707, and HSR4709. These hybrids were produced from the cross between the homozygous PMR line H7B-1Lop-1-1-1, which contained the mutant allele HSPMR of the present invention, and a commercially available powdery mildew susceptible male. Three commercial varieties, Merlin, Magic Lantern and Spartan, which were considered the best commercially available powdery mildew resistant varieties at that time, were used for comparison. A completely randomized experimental design with two replications was used for each treatment and each treatment consisted of 60 plants grown 36 inches apart within a row covered with plastic mulch and 80 inches between the rows. An underground drip irrigation system accompanying a rotten fertilizer supply was used. The trial time period started with direct seeding on May 15, 2005 and was kept as long as possible to allow for a full expression of powdery mildew resistance and plant growth potential in all the tested varieties.

In Table 3, the results of the powdery mildew resistance trial for 2005 are shown. Column 1 shows the variety, column 2 shows the replication, column 3 shows the mean powdery mildew resistance reading based on the scores of 30 plants per replication, and column 4 shows the F test result on the transformed mean of the average powdery mildew resistance reading. The overall transformed means bearing the same letters were not significantly different at the 1% level.

As shown in Table 3, the three varieties containing the mutant allele HSPMR of the present invention had mean scores of about 1.3 while the three commercially available varieties had mean scores of about 3.1. This indicates that the varieties containing the mutant allele HSPMR were highly resistant to powdery mildew whereas the commercial varieties were moderately resistant.

TABLE 3

| Variety | Rep | Mean | Trans Mean |
|---|---|---|---|
| HSR4706 | I | 1.2833 | 0.7792 |
| | II | 1.3167 | 0.7595 |
| | Overall | 1.3000 | 0.7694a |
| HSR4707 | I | 1.3667 | 0.7317 |
| | II | 1.2833 | 0.7792 |
| | Overall | 1.3334 | 0.7505a |
| HSR4709 | I | 1.3333 | 0.7500 |
| | II | 1.3500 | 0.7407 |
| | Overall | 1.3417 | 0.7454a |
| Spartan | I | 2.9667 | 0.3371 |
| | II | 3.0333 | 0.3297 |
| | Overall | 3.0000 | 0.3334b |
| Magic Lantern | I | 3.1333 | 0.3192 |
| | II | 3.1500 | 0.3175 |
| | Overall | 3.1417 | 0.3183b |
| Merlin | I | 3.1500 | 0.3175 |
| | II | 3.3000 | 0.3030 |
| | Overall | 3.225 | 0.3102b |

The second pumpkin trial for powdery mildew resistance was carried out in the summer of 2006 and had the same experimental design and field lay-out. This time, there was no plastic mulch and no drip system was used. Instead, furrow irrigation was used for the trial so that it could make the ground surface as moist as possible to cause the occurrence of powdery mildew.

The level of powdery mildew on plants was divided into six scores or fractions thereof consisting of 0, 1, 2, 3, 4, 5, in which 0 indicates that the plants were completely resistant to the disease and there were no symptoms of the disease on the plants at all, and a rating of 5 indicates that the plants were 100% infected. Increasing one level among the six scores, means that the resistance to the disease decreased by 20% or, conversely, the susceptibility to the disease increased by 20%. For example, a plant with a score of 1 means that plant showed 20% more disease symptoms than a plant with a score of 0. In another example, a plant with a score of 3 means that plant shows 60% more disease symptoms than a plant with a score of 0. Each score is based on the following: the ratio of infected leaves and stems on a plant, the proportion of infected area on a leaf and a stem, and the colony counts out of the total in the infected area. To better understand the difference among the PMR levels, a significance test reciprocal transformation was applied during the mean separation process.

In Table 4, the results of the powdery mildew resistance trial for 2006 are shown. Column 1 shows the variety, column 2 shows the replication, column 3 shows the mean powdery mildew resistance reading based on the scores of 30 plants per replication, and column 4 shows the F test result on the transformed mean of the average powdery mildew resistance reading. The overall transformed means bearing the same letters were not significantly different at the 1% level.

As shown in Table 4, the three varieties containing the mutant allele HSPMR had mean powdery mildew resistance scores of about 1.4 while the commercially available varieties had mean scores of about 3.4. This indicates that the varieties containing the mutant allele HSPMR were highly resistant to powdery mildew whereas the commercial varieties were moderately resistant. These scores also show that the mutant allele HSPMR continued to provide the plants containing the allele with powdery mildew resistance under increased powdery mildew pressure whereas the commercially available varieties were more heavily affected by powdery mildew.

TABLE 4

| Variety | Rep | Mean | Trans Mean |
|---|---|---|---|
| HSR4706 | I | 1.3833 | 0.7229 |
| | II | 1.4167 | 0.7059 |
| | Overall | 1.3917 | 0.7188a |
| HSR4707 | I | 1.4333 | 0.6977 |
| | II | 1.3833 | 0.7229 |
| | Overall | 1.4083 | 0.7013a |
| HSR4709 | I | 1.4167 | 0.7059 |
| | II | 1.4500 | 0.6897 |
| | Overall | 1.4250 | 0.7018a |
| Spartan | I | 3.2500 | 0.3077 |
| | II | 3.2667 | 0.3061 |
| | Overall | 3.2583 | 0.3069b |
| Magic Lantern | I | 3.3667 | 0.2970 |
| | II | 3.3833 | 0.2956 |
| | Overall | 3.3750 | 0.2963b |
| Merlin | I | 3.3833 | 0.2956 |
| | II | 3.4500 | 0.2899 |
| | Overall | 3.4167 | 0.2927b |

The powdery mildew screening tests showed that all of the pumpkin plants containing the mutant allele HSPMR of the present invention unexpectedly had much greater resistance than any of the commercial pumpkin varieties in the two trials. Unexpectedly, when the commercial pumpkin varieties began to die by the beginning of September because of powdery mildew infection, all of the pumpkin plants containing the mutant allele HSPMR of the present invention continued to grow and bear fruit until the first killing frost occurred. The phenotypic expression of the new powdery mildew mutant allele HSPMR in the new hybrids was unexpectedly found to be more than two times greater than the powdery mildew resistance displayed by the commercial varieties based on the differences between the means in Tables 3 and 4. Statistical results in Tables 3 and 4 show that the powdery mildew resistance levels between the new pumpkin hybrids containing the mutant allele HSPMR of the present invention and the powdery mildew resistance levels of the commercial pumpkin varieties were significantly different (F_{0.01}). The powdery mildew resistance exhibited by the new varieties, which contain the mutant allele HSPMR of the present invention, is significantly better than the powdery mildew resistance exhibited by currently available commercial varieties.

### Example 4

### Determining Genetic Similarity Between the Mutant Allele HSPMR of the Present Invention and That of Three Commercially Available Pumpkin Varieties

The genetic similarity between the mutant allele HSPMR of the present invention and that of three commercially available pumpkin varieties was determined. DNA-based SSR, ISSR and RAPD markers were used to compare the genetic relationships of four pumpkin hybrids in April of 2006. The four pumpkin hybrids were Merlin, Spartan, Frosty, and HSR4709. Merlin and Spartan are commercially available pumpkin hybrids that display a level of powdery mildew resistance, while Frosty is a commercially available pumpkin hybrid which is powdery mildew susceptible and was used as a control. HSR4709 is a pumpkin hybrid containing the mutant allele HSPMR of the present invention. Seeds of the four hybrids were planted and germinated in a greenhouse for the purpose of leaf sampling for DNA extraction. Approximately five weeks post germination date about 40 young leaves from each variety were pooled and placed into sterile 1.5 ml microcentrifuge tubes. Tubes containing the leaf samples were freeze-dried overnight with caps open. Genomic DNA was isolated from each entry using a modified CTAB method, quantified by a DNA Flourometer (Hoefer® DyNA Quant 200, Amersham Pharmacia Biotech, USA) and adjusted to 25 ng/μl in sterile TE buffer.

Three different sets of oligonucleotide primers were used to amplify genomic DNA of each entry with minor modifications. All PCR amplifications were carried out in duplicate for accuracy and amplification consistency. Microsatellite markers designed for C. melo (Chiba et al. 2003, Breeding Science 53:21-27), Inter-simple-sequence-repeat (ISSR) primers of 15-23 nucleotides in length (UBC set #9, Biotechnology Laboratory, University of British Columbia, Vancouver, Canada) and Random Amplified Polymorphic DNA Primers (RAPD) of 10 bases in length (Set #9, Genemed Synthesis, Inc. South San Francisco, Calif., USA) were applied for DNA amplification. Microsatellite (SSR) and ISSRs were amplified as described by Chiba et al. (2003) using 160 mM (NH₄)₂SO₄, 670 mM Tris-HCl (pH 8.8 at 25° C.), 0.1% Tween-20, 2.0 mM MgCl₂, 2.5 mM dNTP, 4 μM primer, 25 ng of genomic DNA per 10 μl of reaction volume and 1.0 U of Biolase DNA Polymerase (Bioline USA Inc., Kenilworth, N.J., USA) for 1 cycle denaturation at 94° C. for 3 min, followed by 35 cycles at 94° C. for 60 sec, 45-60° C. for 60 sec, and 72° C. for 1 min, and a 5 min final extension at 72° C. DNA amplification by 10-mer oligonucleotide primers was performed exactly as described above except at an annealing temperature of 35° C. for a total of 40 PCR cycles.

All generated amplicons were separated on 6% polyacrylamide gels. Gels were stained with silver staining method and scored for presence and absence of bands. A locus was considered to be polymorphic if the band was present in one entry and not in the other. The assumption was made that the scored bands were homozygous. Slight distortion of genetic distance could exist by over-estimating genetic similarity with the possibility of the presence of heterozygous bands, which is of greater concern in backcross breeding program than in a database. Monomorphic bands were scored but were not included in any calculations. All of the genotypic data for each entry was scored as a dominant marker.

Coefficients for genetic similarity (GS) between pairs of cultivars were calculated according to Nei and Li (1979):

GS=2Nᵢⱼ/(Nᵢ+Nⱼ)

Where GS equals the similarity coefficient between cultivars i and j, Nᵢⱼ equals the number of common bands present in both i and j cultivars, and Nᵢ and Nⱼ reflect the total of bands detected in cultivars i and j, respectively. GS assessment may attain any value between 0 and 1, where 0 means “no bands in common” and 1 means “patterns are identical”. Consequently, identity of two cultivars will give rise to a GS value of 1, while totally unrelated cultivars will give rise to a GS value of zero. The larger the GS value the more similarities between the entries. It is important to state that a DNA fingerprint of a particular sample is rarely informative on its own. Therefore, as a comparative analysis, the DNA patterns of different samples have to be compared to each other in order to estimate the degree of relatedness. The generated GS values are not absolute and often yield only approximations, becoming intricate to interpret. Such values may be relative to the variability within and between the genetic populations, methodological parameters such DNA marker systems and the statistical methods used for the analysis. For example, a GS value of 0.900 may be interpreted as genetically being the same variety in one species, such as in a highly heterogeneous and obligate open-pollinating crop with an elevated inherent variability within a given variety. In other species, such as in highly homozygous inbred and self-pollinating lines or varieties, a high GS value of 0.980 or 1.00 may be required to demonstrate genetic identity.

Three types of DNA markers were employed in this study, melon microsatellites (SSR), ISSR and RAPD. The genomic DNA of each entry was analyzed in duplicates using 14, 44 and 36 melon microsatellites (SSR), ISSR and RAPD primers, respectively. Repeatable amplifications were produced for most of the primers. In total, the primer combinations yielded 2372 amplification products of which 563 (24%) were polymorphic. The produced and scored polymorphic bands were in general well spaced with easily scoreable bands. The amplification of each entry was carried out in duplicate and no major band discrepancies between reps were noted. Therefore, the scored and highly visible banding pattern under the described amplification conditions of this experiment is considered as being highly repeatable and reliable.

The presence or absence of DNA amplification fingerprint fragments in the pumpkin genotypes considered gave rise to a matrix used to calculate the coefficient of genetic similarity index (GSI) among the four pumpkin lines. The index values indicate the degree of genetic relationship of one entry with another. The index values in Table 5 ranged from 0.324 for hybrids Merlin and HSR4709 to 0.592 for hybrids Spartan and Merlin. As noted the degree of relatedness between the hybrids is described as the genetic similarity index value. For example, the Nei and Li (N&L) genetic similarity index for hybrids Merlin and HSR4709 is 0.324. If this is a truly random sample of DNA markers, which it is, then 95% (i.e. 95% confidence) of random 94 genetic DNA polymorphisms will be between 0.270 and 0.38 confidence limits. In Table 6, LCL means the lower confidence limit at 95% probability level and UCL means the upper confidence limit at 95% probability level.

The results demonstrated that the genetic relationship (GS=0.503) of the new pumpkin HSR4709, which contains the mutant allele HSPMR of the present invention, and PMS pumpkin Frosty were much closer than either of the commercial variety Merlin (GS=0.324) or Spartan (GS=0.362). This shows that the powdery mildew resistance displayed by HSR4709 containing the mutant allele HSPMR of the present invention was derived from a different genetic source from that of either Merlin or Spartan.

TABLE 5

| | Merlin | HSR4709 | Frosty | Spartan |
|---|---|---|---|---|
| Merlin | 1.000 | 0.324 | 0.370 | 0.592 |
| HSR4709 | 0.324 | 1.000 | 0.503 | 0.362 |
| Frosty | 0.370 | 0.503 | 1.000 | 0.467 |
| Spartan | 0.592 | 0.362 | 0.467 | 1.000 |

TABLE 6

| | | LCL | UCL |
|---|---|---|---|
| Merlin | HSR4709 | 0.27 | 0.38 |
| Merlin | Frosty | 0.31 | 0.43 |
| Merlin | Spartan | 0.53 | 0.65 |
| HSR4709 | Frosty | 0.45 | 0.56 |
| HSR4709 | Spartan | 0.31 | 0.42 |
| Frosty | Spartan | 0.41 | 0.53 |

### Further Embodiments of the Invention

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes”. Over the last fifteen to twenty years several methods for producing transgenic plants have been developed and the present invention, in particular embodiments, also relates to transformed versions of the claimed variety or line.

Plant transformation involves the construction of an expression vector which will function in plant cells. Such a vector comprises DNA comprising a gene under control of, or operatively linked to, a regulatory element (for example, a promoter). The expression vector may contain one or more such operably linked gene/regulatory element combinations. The vector(s) may be in the form of a plasmid and can be used alone or in combination with other plasmids to provide transformed C. pepo plants using transformation methods as described below to incorporate transgenes into the genetic material of the C. pepo plant(s).

Expression Vectors for C. pepo Transformation: Marker Genes

Expression vectors include at least one genetic marker operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin. Fraley et al., Proc. Natl. Acad. Sci. USA, 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase and aminoglycoside-3′-adenyl transferase, the bleomycin resistance determinant (Hayford et al., Plant Physiol. 86:1216 (1988), Jones et al., Mol. Gen. Genet., 210:86 (1987), Svab et al., Plant Mol. Biol. 14:197 (1990), Hille et al., Plant Mol. Biol. 7:171 (1986)). Other selectable marker genes confer resistance to herbicides such as glyphosate, glufosinate or bromoxynil (Comai et al., Nature 317:741-744 (1985), Gordon-Kamm et al., Plant Cell 2:603-618 (1990) and Stalker et al., Science 242:419-423 (1988)).

Selectable marker genes for plant transformation not of bacterial origin include, for example, mouse dihydrofolate reductase, plant 5-enolpyruvylshikimate-3-phosphate synthase and plant acetolactate synthase (Eichholtz et al., Somatic Cell Mol. Genet. 13:67 (1987), Shah et al., Science 233:478 (1986), Charest et al., Plant Cell Rep. 8:643 (1990)).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include β-glucuronidase (GUS), β-galactosidase, luciferase and chloramphenicol acetyltransferase (Jefferson, R. A., Plant Mol. Biol. Rep. 5:387 (1987), Teeri et al., EMBO J. 8:343 (1989), Koncz et al., Proc. Natl. Acad. Sci. USA 84:131 (1987), DeBlock et al., EMBO J. 3:1681 (1984)).

In vivo methods for visualizing GUS activity that do not require destruction of plant tissue are available (Molecular Probes publication 2908, IMAGENE GREEN, p. 1-4 (1993) and Naleway et al., J. Cell Biol. 115:151a (1991)). However, these in vivo methods for visualizing GUS activity have not proven useful for recovery of transformed cells because of low sensitivity, high fluorescent backgrounds and limitations associated with the use of luciferase genes as selectable markers.

More recently, a gene encoding Green Fluorescent Protein (GFP) has been utilized as a marker for gene expression in prokaryotic and eukaryotic cells (Chalfie et al., Science 263:802 (1994)). GFP and mutants of GFP may be used as screenable markers.

Expression Vectors for C. pepo Transformation: Promoters

Genes included in expression vectors must be driven by a nucleotide sequence comprising a regulatory element, for example, a promoter. Several types of promoters are well known in the transformation arts as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters that initiate transcription only in a certain tissue are referred to as “tissue-specific”. A “cell-type” specific promoter primarily drives expression in certain cell types in one or more organs, for example, vascular cells in roots or leaves. An “inducible” promoter is a promoter which is under environmental control. Examples of environmental conditions that may effect transcription by inducible promoters include anaerobic conditions or the presence of light. Tissue-specific, tissue-preferred, cell type specific, and inducible promoters constitute the class of “non-constitutive” promoters. A “constitutive” promoter is a promoter that is active under most environmental conditions.

A. Inducible Promoters—An inducible promoter is operably linked to a gene for expression in C. pepo. Optionally, the inducible promoter is operably linked to a nucleotide sequence encoding a signal sequence which is operably linked to a gene for expression in C. pepo. With an inducible promoter the rate of transcription increases in response to an inducing agent.

Any inducible promoter can be used in the instant invention. See Ward et al., Plant Mol. Biol. 22:361-366 (1993). Exemplary inducible promoters include, but are not limited to, that from the ACEI system which responds to copper (Mett et al., Proc. Natl. Acad. Sci. USA 90:4567-4571 (1993)); ln2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone (Schena et al., Proc. Natl. Acad. Sci. USA 88:0421 (1991)).

B. Constitutive Promoters—A constitutive promoter is operably linked to a gene for expression in C. pepo or the constitutive promoter is operably linked to a nucleotide sequence encoding a signal sequence which is operably linked to a gene for expression in C. pepo.

Many different constitutive promoters can be utilized in the instant invention. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2: 163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)). The ALS promoter, Xba1/Ncol fragment 5′ to the Brassica napus ALS3 structural gene (or a nucleotide sequence similarity to said Xba1/Ncol fragment), represents a particularly useful constitutive promoter. See PCT application WO 96/30530.

C. Tissue-specific or Tissue-preferred Promoters—A tissue-specific promoter is operably linked to a gene for expression in C. pepo. Optionally, the tissue-specific promoter is operably linked to a nucleotide sequence encoding a signal sequence which is operably linked to a gene for expression in C. pepo. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter such as that from the phaseolin gene (Murai et al., Science 23:476-482 (1983) and Sengupta-Gopalan et al., Proc. Natl. Acad. Sci. USA 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Signal Sequences for Targeting Proteins to Subcellular Compartments

Transport of protein produced by transgenes to a subcellular compartment such as the chloroplast, vacuole, peroxisome, glyoxysome, cell wall or mitochondrion or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine during protein synthesis and processing where the encoded protein is ultimately compartmentalized.

The presence of a signal sequence directs a polypeptide to either an intracellular organelle or subcellular compartment or for secretion to the apoplast. Many signal sequences are known in the art. See, for example, Becker et al., Plant Mol. Biol. 20:49 (1992); Close, P. S., Master's Thesis, Iowa State University (1993); Knox, C., et al., Plant Mol. Biol. 9:3-17 (1987); Lerner et al., Plant Physiol. 91:124-129 (1989); Frontes et al., Plant Cell 3:483-496 (1991); Matsuoka et al., Proc. Natl. Acad. Sci. 88:834 (1991); Gould et al., J. Cell. Biol. 108:1657 (1989); Creissen et al., Plant J. 2:129 (1991); Kalderon, et al., Cell 39:499-509 (1984); Steifel, et al., Plant Cell 2:785-793 (1990).

### Foreign Protein Genes and Agronomic Genes

With transgenic plants according to the present invention, a foreign protein can be produced in commercial quantities. Thus, techniques for the selection and propagation of transformed plants, which are well understood in the art, yield a plurality of transgenic plants which are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

According to a preferred embodiment, the transgenic plant provided for commercial production of foreign protein is a C. pepo plant. In another preferred embodiment, the biomass of interest is seed. For the relatively small number of transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology, CRC Press, Boca Raton 269:284 (1993). Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR and sequencing, all of which are conventional techniques.

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:

1. Genes that Confer Resistance to Pests or Disease and that Encode:

A. Plant disease resistance genes. Plant defenses are often activated by specific interaction between the product of a disease resistance gene (R) in the plant and the product of a corresponding avirulence (Avr) gene in the pathogen. A plant variety can be transformed with one or more cloned resistance genes to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al. Cell 78:1089 (1994) (Arabidopsis RSP2 gene for resistance to Pseudomonas syringae).

B. A gene conferring resistance to a pest, such as soybean cyst nematode. See e.g., PCT Application WO 96/30517; PCT Application WO 93/19181.

C. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995 and 31998.

D. A lectin. See, for example, Van Damme et al., Plant Molec. Biol. 24:25 (1994), who disclose the nucleotide sequences of several Clivia miniata mannose-binding lectin genes.

E. A vitamin-binding protein such as avidin. See PCT application Ser. No. US 93/06487 which teaches the use of avidin and avidin homologues as larvicides against insect pests.

F. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor I), Sumitani et al., Biosci. Biotech. Biochem. 57:1243 (1993) (nucleotide sequence of Streptomyces nitrosporeus α-amylase inhibitor) and U.S. Pat. No. 5,494,813 (Hepher and Atkinson, issued Feb. 27,1996).

G. An insect-specific hormone or pheromone such as an ecdysteroid or juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

H. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor), and Pratt et al., Biochem. Biophys. Res. Comm. 163:1243 (1989) (an allostatin is identified in Diploptera puntata). See also U.S. Pat. No. 5,266,317 to Tomalski et al., which discloses genes encoding insect-specific, paralytic neurotoxins.

I. An insect-specific venom produced in nature by a snake, a wasp, etc. For example, see Pang et al., Gene 116:165 (1992), for disclosure of heterologous expression in plants of a gene coding for a scorpion insectotoxic peptide.

J. An enzyme responsible for a hyperaccumulation of a monoterpene, a sesquiterpene, a steroid, hydroxamic acid, a phenylpropanoid derivative or another non-protein molecule with insecticidal activity.

K. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 (Scott et al.), which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hornworm chitinase, and Kawalleck et al., Plant Molec. Biol. 21:673 (1993), who provide the nucleotide sequence of the parsley ubi4-2 polyubiquitin gene.

L. A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

M. A hydrophobic moment peptide. See PCT application WO 95/16776, which discloses peptide derivatives of tachyplesin which inhibit fungal plant pathogens, and PCT application WO 95/18855 which teaches synthetic antimicrobial peptides that confer disease resistance.

N. A membrane permease, a channel former or a channel blocker. For example, see the disclosure of Jaynes et al., Plant Sci 89:43 (1993), of heterologous expression of a cecropin-β lytic peptide analog to render transgenic tobacco plants resistant to Pseudomonas solanacearum.

O. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28:451 (1990). Coat protein-mediated resistance has been conferred upon transformed plants against alfalfa mosaic virus, cucumber mosaic virus and tobacco mosaic virus.

P. An insect-specific antibody or an immunotoxin derived therefrom. Thus, an antibody targeted to a critical metabolic function in the insect gut would inactivate an affected enzyme, killing the insect. See Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments).

Q. A virus-specific antibody. See, for example, Tavladoraki et al., Nature 366:469 (1993), who show that transgenic plants expressing recombinant antibody genes are protected from virus attack.

R. A developmental-arrestive protein produced in nature by a pathogen or a parasite. Thus, fungal endo-α-1,4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

S. A developmental-arrestive protein produced in nature by a plant. For example, Logemann et al., Bio/Technology 10:305 (1992), have shown that transgenic plants expressing the barley ribosome-inactivating gene have an increased resistance to fungal disease.

T. Genes involved in the Systemic Acquired Resistance (SAR) Response and/or the pathogenesis-related genes. Briggs, S., Current Biology, 5(2) (1995).

U. Antifungal genes. See Cornelissen and Melchers, Plant Physiol., 101:709-712 (1993); Parijs et al., Planta 183:258-264 (1991) and Bushnell et al., Can. J. of Plant Path. 20(2):137-149 (1998).

V. Genes that confer resistance to Phytophthora root rot, such as the Rps 1, Rps 1-a, Rps 1-b, Rps 1-c, Rps 1-d, Rps 1-e, Rps 1-k, Rps 2, Rps 3-a, Rps 3-b, Rps 3-c, Rps 4, Rps 5, Rps 6, Rps 7 and other Rps genes. See, for example, Shoemaker et al., Phytophthora Root Rot Resistance Gene Mapping in Soybean, Plant Genome IV Conference, San Diego, Calif. (1995).

### 2. Genes That Confer Resistance to an Herbicide, for Example:

A. An herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988), and Miki et al., Theor. Appl. Genet. 80:449 (1990), respectively.

B. Glyphosate (resistance conferred by mutant 5-enolpyruvishikimate-3-phosphate synthase (EPSPS) and aroA genes, respectively) and other phosphono compounds such as glufosinate (phosphinothricin acetyl transferase (PAT) and Streptomyces hygroscopicus PAT bar genes), and pyridinoxy or phenoxy proprionic acids and cyclohexones (ACCase inhibitor-encoding genes). See, for example, U.S. Pat. No. 4,940,835 to Shah, et al., which discloses the nucleotide sequence of a form of EPSPS which can confer glyphosate resistance. A DNA molecule encoding a mutant aroA gene can be obtained under ATCC accession number 39256, and the nucleotide sequence of the mutant gene is disclosed in U.S. Pat. No. 4,769,061 to Comai. European patent application No. 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a PAT gene is provided in European application No. 0 242 246 to Leemans et al. DeGreef et al., Bio/Technology 7:61 (1989) describe the production of transgenic plants that express chimeric bar genes coding for phosphinothricin acetyl transferase activity. Exemplary of genes conferring resistance to phenoxy proprionic acids and cyclohexones, such as sethoxydim and haloxyfop are the Acc1-S1, Acc1-S2, and Acc2-S3 genes described by Marshall et al., Theor. AppL Genet. 83:435 (1992).

C. An herbicide that inhibits photosynthesis, such as a triazine (psbA and gs+ genes) and a benzonitrile (nitrilase gene). Przibila et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker and DNA molecules containing these genes are available under ATCC Accession Nos. 53435, 67441 and 67442. Cloning and expression of DNA coding for a glutathione S-transferase is described by Hayes et al., Biochem. J. 285:173 (1992).

D. Acetohydroxy acid synthase, which has been found to make plants that express this enzyme resistant to multiple types of herbicides, has been introduced into a variety of plants. See Hattori et al., Mol. Gen. Genet. 246:419, 1995. Other genes that confer tolerance to herbicides include a gene encoding a chimeric protein of rat cytochrome P4507A1 and yeast NADPH-cytochrome P450 oxidoreductase (Shiota et al., Plant Physiol., 106:17, 1994), genes for glutathione reductase and superoxide dismutase (Aono et al., Plant Cell Physiol. 36:1687, 1995), and genes for various phosphotransferases (Datta et al., Plant Mol. Biol. 20:619, 1992).

E. Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306; 6,282,837; 5,767,373; and international publication WO 01/12825.

3. Genes that Confer or Contribute to a Value-Added Trait, Such as:

A. Modified fatty acid metabolism, for example, by transforming a plant with an antisense gene of stearyl-ACP desaturase to increase stearic acid content of the plant. See Knultzon et al., Proc. Natl. Acad. Sci. USA 89:2625 (1992).

B. Decreased phytate content—1) Introduction of a phytase-encoding gene enhances breakdown of phytate, adding more free phosphate to the transformed plant. For example, see Van Hartingsveldt et al., Gene 127:87 (1993), for a disclosure of the nucleotide sequence of an Aspergillus niger phytase gene. 2) A gene could be introduced that reduced phytate content. This could be accomplished by cloning and then reintroducing DNA associated with the single allele which is responsible for maize mutants characterized by low levels of phytic acid. See Raboy et al., Maydica 35:383 (1990).

C. Modified carbohydrate composition effected, for example, by transforming plants with a gene coding for an enzyme that alters the branching pattern of starch. See Shiroza et al., J. Bacteriol. 170:810 (1988) (nucleotide sequence of Streptococcus mutants fructosyltransferase gene), Steinmetz et al., Mol. Gen. Genet. 20:220 (1985) (nucleotide sequence of Bacillus subtilis levansucrase gene), Pen et al., Bio/Technology 10:292 (1992) (production of transgenic plants that express Bacillus lichenifonnis α-amylase), Elliot et al., Plant Molec. Biol. 21:515 (1993) (nucleotide sequences of tomato invertase genes), Søgaard et al., J. Biol. Chem. 268:22480 (1993) (site-directed mutagenesis of barley α-amylase gene), and Fisher et al., Plant Physiol. 102:1045 (1993) (maize endosperm starch branching enzyme II).

D. Elevated oleic acid via FAD-2 gene modification and/or decreased linolenic acid via FAD-3 gene modification. See U.S. Pat. Nos. 6,063,947; 6,323,392; and international publication WO 93/11245.

4. Genes that Control Male Sterility

A. Introduction of a deacetylase gene under the control of a tapetum-specific promoter and with the application of the chemical N—Ac—PPT. See international publication WO 01/29237.

B. Introduction of various stamen-specific promoters. See international publications WO 92/13956 and WO 92/13957.

C. Introduction of the barnase and the barstar genes. See Paul et al., Plant Mol. Biol. 19:611-622, 1992).

Methods for C. pepo Transformation

Numerous methods for plant transformation have been developed including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc. Boca Raton, 1993) pages 67-88. In addition, expression vectors and in-vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

### A. Agrobacterium-Mediated Transformation

One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra and Moloney et al., Plant Cell Reports 8:238 (1989). See also, U.S. Pat. No. 5,563,055 (Townsend and Thomas), issued Oct. 8, 1996.

### B. Direct Gene Transfer

Several methods of plant transformation, collectively referred to as direct gene transfer, have been developed as an alternative to Agrobacterium-mediated transformation. A generally applicable method of plant transformation is microprojectile-mediated transformation where DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. Sanford et al., Part. Sci. Technol. 5:27 (1987); Sanford, J. C., Trends Biotech. 6:299 (1988); Klein et al., Bio/Tech. 6:559-563 (1988); Sanford, J. C. Physiol Plant 7:206 (1990); Klein et al., Biotechnology 10:268 (1992). See also U.S. Pat. No. 5,015,580 (Christou, et al.), issued May 14, 1991 and U.S. Pat. No. 5,322,783 (Tomes, et al.), issued Jun. 21, 1994.

Another method for physical delivery of DNA to plants is sonication of target cells. Zhang et al., Bio/Technology 9:996 (1991). Alternatively, liposome and spheroplast fusion have been used to introduce expression vectors into plants. Deshayes et al., EMBO J., 4:2731 (1985); Christou et al., Proc Natl. Acad. Sci. USA 84:3962 (1987). Direct uptake of DNA into protoplasts using CaCl₂ precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described (Donn et al., In Abstracts of VIIth International Congress on Plant Cell and Tissue Culture IAPTC, A2-38, p 53 (1990); D'Halluin et al., Plant Cell 4:1495-1505 (1992) and Spencer et al., Plant Mol. Biol. 24:51-61 (1994)).

Following transformation of C. pepo target tissues, expression of the above-described selectable marker genes allows for preferential selection of transformed cells, tissues and/or plants, using regeneration and selection methods well known in the art.

The foregoing methods for transformation would typically be used for producing a transgenic variety. The transgenic variety could then be crossed with another (non-transformed or transformed) variety in order to produce a new transgenic variety. Alternatively, a genetic trait that has been engineered into a particular C. pepo line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties that do not contain that gene. As used herein, “crossing” can refer to a simple X by Y cross or the process of backcrossing depending on the context.

### Single-Gene Conversions

When the term “C. pepo plant” is used in the context of the present invention, this also includes any single gene conversions of that variety. The term single gene converted plant as used herein refers to those C. pepo plants which are developed by a plant breeding technique called backcrossing wherein essentially all of the desired morphological and physiological characteristics of a variety are recovered in addition to the single gene transferred into the variety via the backcrossing technique. Backcrossing methods can be used with the present invention to improve or introduce a characteristic into the variety. The term “backcrossing” as used herein refers to the repeated crossing of a hybrid progeny back to the recurrent parent, i.e., backcrossing 1, 2, 3, 4, 5, 6, 7, 8 or more times to the recurrent parent. The parental C. pepo plant that contributes the gene for the desired characteristic is termed the nonrecurrent or donor parent. This terminology refers to the fact that the nonrecurrent parent is used one time in the backcross protocol and therefore does not recur. The parental C. pepo plant to which the gene or genes from the nonrecurrent parent are transferred is known as the recurrent parent as it is used for several rounds in the backcrossing protocol (Poehlman & Sleper, 1994; Fehr, 1987). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a C. pepo plant is obtained wherein essentially all of the desired morphological and physiological characteristics of the recurrent parent are recovered in the converted plant, in addition to the single transferred gene from the nonrecurrent parent

The selection of a suitable recurrent parent is an important step for a successful backcrossing procedure. The goal of a backcross protocol is to alter or substitute a single trait or characteristic in the original variety. To accomplish this, a single gene of the recurrent variety is modified or substituted with the desired gene from the nonrecurrent parent, while retaining essentially all of the rest of the desired genetic, and therefore the desired physiological and morphological, constitution of the original variety. The choice of the particular nonrecurrent parent will depend on the purpose of the backcross; one of the major purposes is to add some agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many single gene traits have been identified that are not regularly selected for in the development of a new variety but that can be improved by backcrossing techniques. Single gene traits may or may not be transgenic; examples of these traits include but are not limited to, male sterility, waxy starch, herbicide resistance, resistance for bacterial, fungal, or viral disease, insect resistance, male fertility, enhanced nutritional quality, industrial usage, yield stability and yield enhancement. These genes are generally inherited through the nucleus. Several of these single gene traits are described in U.S. Pat. Nos. 5,959,185; 5,973,234 and 5,977,445; the disclosures of which are specifically hereby incorporated by reference.

### Tissue Culture

Further reproduction of a variety can occur by tissue culture and regeneration. Tissue culture of various tissues of C. pepo and regeneration of plants therefrom is well known and widely published. For example, reference may be had to Jelaska, S. et al., Physiol. Plant. 64(2):237-242 (1985) and Krsnik-Rasol, M., Int. J. Dev. Biol. 35(3):259-263 (1991). Thus, another aspect of this invention is to provide cells which upon growth and differentiation produce C. pepo plants having the mutant allele HSPMR.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, pods, leaves, stems, roots, root tips, anthers, pistils and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference.

### Additional Breeding Methods

This invention also is directed to methods for producing a C. pepo plant by crossing a first parent C. pepo plant with a second parent C. pepo plant wherein the first or second parent C. pepo plant is a C. pepo plant comprising the mutant allele HSPMR. Further, both first and second parent C. pepo plants can comprise the mutant allele HSPMR. Thus, any such methods using a C. pepo plant comprising the mutant allele HSPMR are part of this invention: selfing, backcrosses, hybrid production, crosses to populations, and the like.

### DEPOSIT INFORMATION

Pumpkin seeds containing the HSPMR mutant allele have been placed on deposit with the American Type Culture Collection (ATCC), 10801 University Boulevard, Manassas, Va. 20110-2209 on Jan. 23, 2007 and having Deposit Accession Number PTA-8167.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A C. pepo seed containing an allele designated HSPMR, wherein a representative sample of seed containing said allele HSPMR was deposited under ATCC Accession No. PTA-8167.

2. A C. pepo plant, or a part thereof, produced by growing said C. pepo seed of claim 1.

3. A tissue culture of cells produced from the plant of claim 2, wherein said cells of the tissue culture are produced from a plant part selected from the group consisting of leaf, pollen, embryo, cotyledon, hypocotyl, meristematic cell, root, root tip, pistil, anther, flower, stem, fruit and petiole.

4. A protoplast produced from the plant of claim 2.

5. A protoplast produced from the tissue culture of claim 3.

6. A C. pepo plant regenerated from the tissue culture of claim 3.

7. A method for producing an F₁ hybrid C. pepo seed, wherein the method comprises crossing the plant of claim 2 with a different C. pepo plant and harvesting the resultant F₁ hybrid C. pepo seed.

8. A hybrid C. pepo seed produced by the method of claim 7.

9. A hybrid C. pepo plant, or a part thereof, produced by growing said hybrid seed of claim 8.

10. A method of producing an herbicide resistant C. pepo plant, wherein the method comprises transforming the C. pepo plant of claim 2 with a transgene wherein the transgene confers resistance to an herbicide selected from the group consisting of imidazolinone, sulfonylurea, glyphosate, glufosinate, L-phosphinothricin, triazine and benzonitrile.

11. An herbicide resistant C. pepo plant produced by the method of claim 10.

12. A method of producing an insect resistant C. pepo plant, wherein the method comprises transforming the C. pepo plant of claim 2 with a transgene that confers insect resistance.

13. An insect resistant C. pepo plant produced by the method of claim 12.

14. The C. pepo plant of claim 13, wherein the transgene encodes a Bacillus thuringiensis endotoxin.

15. A method of producing a disease resistant C. pepo plant, wherein the method comprises transforming the C. pepo plant of claim 2 with a transgene that confers disease resistance.

16. A disease resistant C. pepo plant produced by the method of claim 15.

17. A method of producing a C. pepo plant with modified fatty acid metabolism or modified carbohydrate metabolism, wherein the method comprises transforming the C. pepo plant of claim 2 with a transgene encoding a protein selected from the group consisting of fructosyltransferase, levansucrase, α-amylase, invertase and starch branching enzyme or encoding an antisense of stearyl-ACP desaturase.

18. A C. pepo plant having modified fatty acid metabolism or modified carbohydrate metabolism produced by the method of claim 17.
